(19) United States
(12) Patent Application Publication
(10) Pub. No.: US 2003/0104427 A1
(43) Pub. Date: Jun. 5, 2003

(54) Methods to modulate telomere structure and function

(76) Inventors: John H.J. Petrini, New York, NY (US); Titia de Lange, New York, NY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/176,098

(22) Filed: Jun. 20, 2002

Related U.S. Application Data
(60) Provisional application No. 60/300,705, filed on Jun. 25, 2001.

(51) Int. Cl. C12Q 1/68; A61K 31/00

(52) U.S. Cl. 435/6; 514/1

(57) ABSTRACT

A method to detect cells in S phase and a method to modulate telomere function are provided.

### CROSS-REFERENCE TO RELATED APPLICATIONS

This application claims the benefit of the filing date of U.S. application Serial No. 60/300,705, filed on Jun. 25, 2001, under 35 U.S.C. §119(e).

### STATEMENT OF GOVERNMENT RIGHTS

This invention was made with a grant from the Government of the United States of America (grant GM56888 from the National Institutes of Health). The Government may have certain rights in the invention.

### BACKGROUND OF THE INVENTION

Nijmegen Breakage Syndrome (NBS) is a rare autosomal recessive disorder characterized by microcephaly, growth retardation, immunodeficiency, and an increased incidence of cancer, e.g., hematopoietic malignancy (van der Burgt et al., 1996; and Weemaes et al., 1981). At the cellular level, NBS is characterized by cell cycle defects and radiation sensitivity. The clinical features of NBS overlap to some extent with those of ataxia telangiectasia (AT); thus it has been described as an AT variant syndrome (Saar et al., 1997). However, NBS is genetically distinct. The two syndromes are clinically distinguishable in that NBS patients do not exhibit neurological abnormalities, telangiectasia or increased α-feto protein levels observed in AT patients (reviewed in Shiloh, 1997).

Nonetheless, NBS and AT exhibit remarkably similar phenotypes at the cellular level, suggesting that the corresponding gene products function in the same pathway. Unlinked non-complementation of chromosome instability was observed in heterokaryons of AT and NBS fibroblasts, leading to speculation that the respective gene products are physically associated (Stumm et al., 1997). Cells from both NBS and AT patients show increased sensitivity to ionizing radiation (IR) as well as increased levels of spontaneous and induced chromosomal fragility. In addition, NBS and AT cells fail to induce p53 at the G1/S checkpoint, and fail to suppress DNA synthesis in response to ionizing radiation (radioresistant DNA synthesis) (Jongmans et al., 1997; Perez-Vera et al., 1997; Sullivan et al., 1997; Taalman et al., 1983; and Young and Painter, 1989) (AT reviewed in Hoekstra, 1997; and Shiloh, 1997). Together, these data suggest that the AT gene product, ATM, is a component of, or functions in close proximity to, the primary sensor of DNA damage.

Accordingly, AT phenotypes can be explained by the failure to signal the presence of DNA damage. Hence, IR sensitivity in AT cells is generally attributed to defects in the cellular DNA damage response. However, some data suggest that DNA repair functions in AT cells may also be affected (Blocher et al., 1991; Cornforth and Bedford, 1985; Murnane, 1995; and Pandita and Hittelman, 1992). Consistent with this notion, cells established from AT patients exhibit increased rates of intrachromosomal DNA recombination (Meyn, 1993).

DNA damage includes double strand breaks in the DNA. These breaks in DNA are repaired by the double strand break (DSB) repair complex. The human DSB complex, also referred to as the “hMre11/hRad50 complex” or the Mre11 complex or the MRN complex, was shown to consist of five proteins: hMre11, hRad50, and three additional proteins of 95 kDa, 200 kDa, and 400 kDa (Dolganov et al., 1996; Petrini et al., 1995). The phenotypic features of yeast lacking the counterparts to hMre11 and hRad50, i.e., Scmre11 and Scrad50 mutants, include hyperrecombination, sensitivity to DNA damaging agents, and DNA repair deficiency (Ajimura et al., 1993; and Game, 1993). These features are reminiscent of chromosomal instability syndromes such as AT, NBS, Bloom syndrome and others (Ellis, 1997; Fukuchi et al., 1989; Gatti et al., 1991; Meyn, 1995; and van der Burgt et al., 1996). The conservation of Mre11 and Rad50 functions predicts that similar phenotypic outcomes would result from mutations in humans that affect the hMre11/hRad50 protein complex.

Recently, Carney et al. (1998) identified DNA encoding human p95 (Nbs1) and showed that human p95 is associated with the hRad50/hMre11 complex and the damage response to radiation. However, it is unclear whether this complex, or any of its proteins, is associated with other cellular processes, e.g., telomere function.

### SUMMARY OF THE INVENTION

The invention provides a method to identify an agent that modulates telomere function, e.g., length or maintenance including repair or replication, which is associated with the Mre11 complex. The method comprises contacting a mammalian cell or a subcellular portion thereof, e.g., isolated nuclei, an extract thereof, e.g., a nuclear extract, or a composition comprising two or more isolated proteins of the Mre11 complex, and optionally TRF2, and DNA comprising a telomere, with an agent. Then it is detected or determined whether the agent modulates the amount, formation (presence) or activity of the Mre11 complex at telomeres. As used herein, “isolated and/or purified” refers to in vitro preparation, isolation and/or purification of a protein or a complex of biomolecules, e.g., Mre11 complex, so that it is not associated with in vivo substances or is substantially purified from in vitro substances.

As described hereinbelow, the Mre11 complex mediates functions relevant to telomere maintenance and function. For example, nanoelectrospray tandem mass spectrometry revealed the presence of Rad50 protein in TRF2 immunocomplexes. Protein blotting showed that a small fraction of Rad50 as well as Mre11 and the third component of the Mre11 double strand break (DSB) repair complex, the Nijmegen Breakage Syndrome protein (Nbs1), are associated with TRF2. Further, indirect immunofluorescence demonstrated the presence of Rad50 and Mre11 at interphase telomeres, while Nbs1 associated with TRF2 and interphase telomeres in S-phase but not in G1 or G2. And although the Mre11 complex migrated to sites of DNA damage in response to γ-irradiation, TRF2 did not relocate from telomeres in damaged cells and irradiation did not affect the association of TRF2 with the Mre11 complex, arguing against a role for TRF2 in DSB repair. Instead, the Mre11 complex functions at telomeres, possibly by modulating t-loop formation.

In one embodiment of the invention, the agent can be contacted with a cell transformed with an expression cassette comprising a promoter operably linked to a DNA segment encoding TRF2, p95, Mre11, Rad50 or another protein in the Mre11 complex, or a portion thereof such as a peptide, and optionally a corresponding nontransformed (wild type) cell. Alternatively, the agent may be contacted with a cell which lacks, or has reduced levels of, one or more proteins in the Mre11 complex or TRF2. To prepare a recombinant cell which lacks or has reduced levels of one or more proteins in the Mre11 complex or TRF2, an expression cassette having a DNA segment encoding a protein in the Mre11 complex or TRF2 which is in an anti-sense orientation relative to the promoter or a DNA segment that is operatively linked to the promoter in a sense orientation, may be employed to decrease or eliminate transcription of the corresponding endogenous gene for example via antisense inhibition of transcription or via a gene knock-out, respectively. In yet another embodiment, an animal model may be employed to detect an agent that modulates telomere function, e.g., a non-human mammal such as a mouse that has reduced or enhanced levels of one or more proteins in the Mre11 complex. Moreover, an expression cassette of the invention may be employed in a method to modulate telomere length or maintenance in a cell, as described herein.

Thus, once an agent is identified that modulates the amount, formation or activity of the Mre11 complex at telomeres, e.g., a small molecule inhibitor of p95, the invention also provides a method of using the agent to modulate telomere function, e.g., length or maintenance, in a cell or mammal.

The invention further provides a method to detect cells in S phase. The method comprises contacting a mammalian cell, a subcellular fraction of a mammalian cell or an extract of a mammalian cell which comprises DNA comprising a telomere, with an agent that binds p95 so as to form a complex. Then it is detected or determined whether the complex is associated with a telomere.

Also provided is a method to detect telomere replication. The method comprises contacting a mammalian cell, a subcellular fraction of a mammalian cell or an extract of a mammalian cell which comprises DNA comprising a telomere, with an agent that binds p95 so as to form a complex. Then it is detected or determined whether the complex is associated with a telomere.

Further provided is a method to detect t-loop formation in cells. The method comprises contacting a mammalian cell, a subcellular fraction of a mammalian cell or an extract of a mammalian cell which comprises DNA comprising a telomere, with an agent that binds p95 so as to form a complex. Then it is detected or determined whether the complex is associated with TRF2 at a telomere.

The invention also provides therapeutic methods, e.g., a method to prevent or treat a patient having an indication (e.g., a disorder or disease) associated with aberrant or reduced telomere function, e.g., ageing or accelerating ageing, neoplastic disease, regenerative disorders, intestinal disorders or germ cell disorders such as Ataxia telangiectasia, Werner syndrome, Bloom syndrome, Nijmegen breakage syndrome, and dyskeratosis congenita, all of which are linked to telomere function or telomere metabolism defects. For example, agents of the invention which enhance the amount, formation or activity of the Mre11 complex at telomeres are particularly useful in anti-ageing applications or applications in which the inhibition of telomere shortening is indicated, or after exposure of a mammal to a moiety that damages telomere structure or function and, agents of the invention which reduce or inhibit the amount, formation or activity of the Mre11 complex at telomeres, are particularly useful to selectively kill proliferating cells, e.g., cancer cells.

### BRIEF DESCRIPTION OF THE FIGURES

FIG. 1. Association of TRF2 with the Mre11 complex. A) Identification of TRF2 associated proteins. TRF2 immunocomplexes were separated on a 8% SDS-polyacrylamide gel and stained with Coomassie blue. Four bands that were consistently observed are indicated. B) Identification of Rad50 by nanoelectrospray tandem mass spectrometry. Sequencing of nine tryptic peptides unambiguously identified the presence of human Rad50 in the 150 kDa band. The position of the first amino acid of each peptide is given. C) Co-immunoprecipitation of Rad50, Mre11 and Nbs1 with TRF2. Immunoprecipitations of HeLa nuclear extract (500 μg) were carried out using either preimmune serum or anti-TRF2 antibodies Ab#647 or Ab#508. Western blotting was performed with rabbit anti-Rad50, anti-Mre11, and anti-Nbs1, anti-TRF2 (#647), anti-RAP1 (#765) and mouse anti-TRF1 sera (#3; S. Smith and T. de Lange, unpublished). The input lanes contained 20 μg of HeLa nuclear extract. D) Co-immunoprecipitation of TRF2 with Mre11 and Nbs1. Immunoprecipitations of HeLa nuclear extract (500 μg) were carried out using either preimmune serum or anti-Mre11 or anti-Nbs1 antibodies. Western blotting was performed with rabbit anti-TRF2 (#647), anti-Mre11 and anti-Nbs1. The input lanes contained 15 μg of HeLa nuclear extract. E) Co-immunoprecipitation of the Mre11 complex with TRF2 from IMR90 cell extract. Immunoprecipitations were carried out essentially as described in C) except that whole cell extract from young IMR90 cells was used. F) DNA binding activity of TRF2 is not required for association with the Mre11 complex. Immunoprecipitations were carried out using antibody M2 (Sigma) and whole cell extracts (1.0 mg) from HTC75-derived cell lines³ induced to express either FLAG-tagged TRF2^{ΔBΔM}(T4 and T19) or FLAG-tagged TRF₂^{ΔB} (S13) or no exogenous protein (B27). Western blotting was performed on the M2 immunocomplexes with rabbit anti-Rad50, anti-MRE1, and anti-Nbs1. Input lanes contained 40 μg whole cell extracts and lanes marked “IP-M2” contained M2-immunoprecipitates.

FIG. 2. Presence of Rad50, Mre11 and Nbs1 at telomeres. A) Co-localization of Rad50/Mre11/Nbs1 with TRF1 at HeLa telomeres. Interphase HeLaI.2.11 cells were permeabilized with Triton-X-100 and stained with mouse anti-TRF1 antibody (TRITC) in conjunction with relevant rabbit anti-Rad50, anti-Mre11 and anti-Nbs1 antibodies (FITC) as indicated. As a control for bleed through of the FITC signal into the TRITC channel and vice versa, detergent-extracted and fixed cell nuclei were stained with either rabbit anti-Rad50 antibody alone (FITC) or mouse anti-TRF1 antibody alone (TRITC) and processed with both secondary antibodies. B) Co-localization of Rad50/Mre11/Nbs1 proteins with TRF1 in the ALT cell line W138VA13/2RA. Interphase cells were extracted with detergent and stained with the indicated antibodies as described above.

FIG. 3. Cell cycle regulated association of Nbs1 with TRF2 and telomeres. A) FACS analysis of synchronized HeLa cells. Y-axis: cell numbers; X-axis: relative DNA content, based on staining with propidium iodide. 0-10 hrs: cells were released for 0-10 hrs from a thymidine/aphidicolin block. Asyn: asynchronous population. B) Western analysis of steady state levels of Nbs1 and TRF2. Whole cell extract (20 μg) from HeLa cells harvested at the indicated time points was fractionated on SDS-PAGE, blotted and incubated with the indicated primary antibodies. C) Association of Nbs1 with TRF2 in S-phase. Immunoprecipitations were carried out with either preimmune serum (pre) or anti-TRF2 Ab#647 and whole cell extracts of HeLa cells harvested at the indicated time points. TRF2 immunoprecipitates were analyzed by Western blotting with anti-Rad50, anti-Mre11, anti-Nbs1 and anti-TRF2. HNE: 20 μg of HeLa nuclear extract. D) Cell cycle regulated localization of Nbs1 at telomeres. Synchronized HeLa cells from the indicated time points were treated as described in FIG. 2A. The fixed cells were dually stained with rabbit polyclonal anti-Nbs1 antibody (FITC) and mouse anti-TRF1 antibody (TRITC).

FIG. 4. γ-irradiation does not affect the telomeric localization of TRF2 or its interaction with the Mre11 complex. A) Formation of Rad50 foci in γ-irradiated cells and persistence of TRF2 at telomeres. HeLa cells were γ-irradiated at a dose of 12 Gy. Eight-hours post-γ-irradiation cells were fixed in 3% paraformaldehyde and 2% sucrose, followed by permeabilization in Triton X-100 buffer. Non-irradiated cells were used as a control. The fixed cells were either immunostained with anti-Rad50 antibody alone (in green, FITC; top panels) or immunostained anti-TRF2 (#647; FITC) together with a mouse anti-TRF1 antibody (#3; TRITC). DNA was stained with DAPI. B) Gamma irradiation does not affect the association of Rad50/Mre11/Nbs1 and RAP1 with TRF2. Coimmunoprecipitations were carried out with anti-TRF2 antibody (#647) and cell extracts made from non-irradiated HeLa cells or cells harvested at 0 hour, 4 hours, 8 hours, and 22 hours post γ-irradiation. Whole cell extract (30 μg) from either non-irradiated or irradiated cells was loaded as input. The immunoprecipitates and inputs were analyzed by Western blotting with anti-Rad50, anti-Mre11, anti-Nbs1, anti-TRF2 and anti-RAP1 antibodies. A control using preimmune serum (pre) was performed with non-irradiated cell extracts.

### DETAILED DESCRIPTION OF THE INVENTION

To identify agents that specifically interact with one or more proteins of the mammalian Mre11 complex so as to modulate telomere length or maintenance, whole cells, including mutant cells naturally lacking or having reduced amounts of one or more proteins in the Mre11 complex or recombinant cells, the genome of which has been altered by exogenous DNA so as enhance or increase, or alternatively to reduce or eliminate expression of one or more proteins in the Mre11 complex (i.e., either overexpressors or gene knock-outs), portions of cells, e.g., nuclei or extracts such as nuclear extracts, or a mixture of two or more isolated proteins of the Mre11 complex may be employed. The agent may be selcted as one which enhances or alternatively one which inhibits the formation, amount or activity of the Mre11 complex at telomeres. In particular, inhibitors and enhancers of Nbs1 (p95), the interaction of p95 or other proteins in the Mre11 complex and telomeres, and the interaction of p95 and Mre11, are of interest. For example, peptides of one of the proteins of the Mre11 complex may be employed to identify peptides which specifically bind to other proteins in the complex or to TRF2, which can be the basis for the preparation of antibodies, or the design or identification of small molecules, which inhibit or enhance Nbs1 or the interaction of p95 or other proteins in the Mre11 complex and telomeres, or p95 and Mre11.

I. Preparation of Biomolecules Useful To Identify Agents of the Invention

A. Nucleic Acid Molecules

1. Chimeric Expression Cassettes

To prepare expression cassettes for transformation, the recombinant DNA sequence or segment may be circular or linear, double-stranded or single-stranded. A recombinant DNA sequence which encodes an RNA sequence that is substantially complementary to a mRNA sequence encoding mammalian TRF2, p95, Mre11, Rad50 or one of the other proteins in the Mre11 complex, or a portion thereof, e.g., a peptide, is typically a “sense” DNA sequence cloned into a cassette in the opposite orientation (i.e., 3′ to 5′ rather than 5′ to 3′). However, as described herein, antisense sequences may also be of use. Generally, the recombinant DNA sequence or segment is in the form of chimeric DNA, such as plasmid DNA, that can also contain coding regions flanked by control sequences which promote the expression of the recombinant DNA present in the resultant cell line.

As used herein, “chimeric” means that a vector comprises DNA from at least two different species, or comprises DNA from the same species, which is linked or associated in a manner which does not occur in the “native” or wild type of the species. In particular, a chimeric vector may include the linking of an open reading frame encoding at least a portion of mammalian TRF2, p95, Mre11, Rad50 or one of the other proteins in the Mre11 complex, with another nucleic acid segment that encodes a peptide, e.g., GST or 6×His, so as to encode a fusion polypeptide. The portion of the fusion polypeptide that is not TRF2, p95, Mre11, Rad50 or one of the other proteins in the Mre11 complex, is useful to isolate the fusion polypeptide from other host cell polypeptides.

Aside from recombinant DNA sequences that serve as transcription units for TRF2, p95, Mre11, Rad50 or one of the other proteins in the Mre11 complex, a portion of the recombinant DNA may be untranscribed, serving a regulatory or a structural function. For example, the recombinant DNA may itself comprise a promoter that is active in mammalian cells, or may utilize a promoter already present in the genome that is the transformation target. Such promoters include the CMV promoter, as well as the SV40 late promoter and retroviral LTRs (long terminal repeat elements), although many other promoter elements well known to the art may be employed in the practice of the invention.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The recombinantly DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinuspyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory Press (2d ed., 1989), provides suitable methods of construction.

2. Transformation into Host Cells

The recombinant DNA can be readily introduced into the host cells, e.g., mammalian, bacterial, yeast or insect cells by transfection with an expression vector comprising DNA encoding mammalian TRF2, p95, Mre11, Rad50 or another protein of the Mre11 complex, or its complement, by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a recombinant DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, it is desirable to use an efficient means of precisely inserting a single copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As used herein, the term “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources. Generally, the recombinant DNA sequence is related to a DNA sequence which is resident in the genome of the host cell but is not expressed, or not highly expressed, or, alternatively, overexpressed.

“Transfected” or “transformed” is used herein to include any host cell or cell line, the genome of which has been altered or augmented by the presence of at least one recombinant DNA sequence, which DNA is also referred to in the art of genetic engineering as “heterologous DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence. Preferably, the transfected DNA is a chromosomally integrated recombinant DNA sequence, which comprises a gene encoding mammalian TRF2, p95, Rad50, Mre11 or one of the other proteins in the Mre11 complex or its complement, which host cell may or may not express significant levels of autologous or “native” TRF2, p95, Rad50, Mre11 or one of the other proteins in the Mre11 complex.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of TRF2, p95, Rad50, Mre11 or one of the other proteins in the Mre11 complex, e.g., by immunological means (immunoprecipitations, immunoaffinity columns, ELISAs and Western blots) or by any other assay useful to identify molecules falling within the scope of the invention.

To detect and quantitate RNA produced from introduced DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the DNA segment in question, they do not provide information as to whether the DNA segment is being expressed. Expression may be evaluated by specifically identifying the peptide products of the introduced DNA sequences or evaluating the phenotypic changes brought about by the expression of the introduced DNA segment in the host cell.

B. Polypeptides

The present isolated, purified polypeptides or peptides, variants or derivatives thereof, can be synthesized in vitro, e.g., by the solid phase peptide synthetic method or by recombinant DNA approaches (see above). The solid phase peptide synthetic method is an established and widely used method, which is described in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides and polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, derivatives, e.g., chemically derived derivatives, of a given TRF2, p95, Rad50, Mre11 or other protein of the Mre11 complex, or a peptide thereof, that modulate to telomere maintenance or length can be readily prepared. For example, amides of TRF2, p95, Rad50, Mre11 or other protein of the Mre11 complex, or a peptide thereof, may also be prepared by techniques well known in the art for converting a carboxylic acid group or precursor, to an amide. A preferred method for amide formation at the C-terminal carboxyl group is to cleave the peptide or polypeptide from a solid support with an appropriate amine, or to cleave in the presence of an alcohol, yielding an ester, followed by aminolysis with the desired amine.

Salts of carboxyl groups of TRF2, p95, Rad50, Mre11 or other protein of the Mre11 complex, or a peptide thereof, may be prepared in the usual manner by contacting the peptide or polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of TRF2, p95, Rad50, Mre11 or other protein of the Mre11 complex, or a peptide thereof, may be prepared by utilizing an N-acyl protected amino acid for the final condensation, or by acylating a protected or unprotected peptide. O-acyl derivatives may be prepared, for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired. Formyl-methionine, pyroglutamine and trimethyl-alanine may be substituted at the N-terminal residue of the peptide or polypeptide. Other amino-terminal modifications include aminooxypentane modifications (see Simmons et al., Science, 276, 276 (1997)).

In addition, the amino acid sequence of TRF2, p95, Mre11, Rad50 or another protein of the Mre11 complex, or a peptide thereof can be modified so as to result in a variant of TRF2, p95, Mre11, Rad50 or another protein of the Mre11 complex. The modification includes the substitution of at least one amino acid residue for another amino acid residue, including substitutions which utilize the D rather than L form, as well as other well known amino acid analogs. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, α-methyl-alanine, para-benzoyl-phenylalanine, phenylglycine, propargylglycine, sarcosine, and tert-butylglycine.

One or more of the residues of the polypeptide can be altered, so long as the polypeptide variant is biologically active. It is preferred that the variant polypeptide has substantially similar activity to that of the corresponding non-variant polypeptide. Conservative amino acid substitutions are preferred—that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic; trp, tyr, phe.

The invention also envisions polypeptide variants with non-conservative substitutions. Non-conservative substitutions entail exchanging a member of one of the classes described above for another.

Acid addition salts of the amino residues of the polypeptide, peptide, or variant thereof may be prepared by contacting the polypeptide, peptide or variant amine with one or more equivalents of the desired inorganic or organic acid, such as, for example, hydrochloric acid. Esters of carboxyl groups of the polypeptides or peptides may also be prepared by any of the usual methods known in the art.

C. Antibodies

Antibodies useful in the invention are prepared by using standard techniques. To prepare polyclonal antibodies or “antisera,” an animal is inoculated with an antigen, i.e., a purified immunogenic TRF2, p95, Mre11, Rad50 or another protein of the Mre11 complex, or a peptide thereof, and immunoglobulins are recovered from a fluid, such as blood serum, that contains the immunoglobulins, after the animal has had an immune response. For inoculation, the antigen is preferably bound to a carrier peptide and emulsified using a biologically suitable emulsifying agent, such as Freund's incomplete adjuvant. A variety of mammalian or avian host organisms may be used to prepare polyclonal antibodies against TRF2, p95, Mre11, Rad50 or another protein of the Mre11 complex, or a peptide thereof.

Following immunization, Ig is purified from the immunized bird or mammal, e.g., goat, rabbit, mouse, rat, or donkey and the like. For certain applications, particularly certain pharmaceutical applications, it is preferable to obtain a composition in which the antibodies are essentially free of antibodies that do not react with the immunogen. This composition is composed virtually entirely of the high titer, monospecific, purified polyclonal antibodies to the immunogen. Antibodies can be purified by affinity chromatography, using purified TRF2, p95, Rad50, Mre11 or another protein of the Mre11 complex, or a peptide thereof. Purification of antibodies by affinity chromatography is generally known to those skilled in the art (see, for example, U.S. Pat. No. 4,533,630). Briefly, the purified antibody is contacted with the purified immunogen bound to a solid support for a sufficient time and under appropriate conditions for the antibody to bind to the immunogen. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically. Immunologically active fragments of the present antibodies are also within the scope of the present invention, e.g., the F(ab) fragment and scFv antibodies, as are partially humanized monoclonal antibodies.

Thus, it will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

The antibodies of the invention are useful for detecting or determining whether an agent of the invention inhibits or enhances telomere function, e.g., length or maintenance, that is associated with the Mre11 complex as well as modulating telomere function length via interactions with one or more proteins of the TRF2-Mre11 complex. The antibodies are contacted with a mammalian sample, e.g., tissue biopsy, mammalian physiological fluid comprising cells, cultured cells, nuclei, or extracts thereof, which have been contacted with a test agent for a period of time and under conditions sufficient for antibodies to bind to TRF2, p95, Mre11, Rad50 or another protein of the Mre11 complex, so as to form a binary complex between at least a portion of said antibodies and said polypeptide. Such times, conditions and reaction media can be readily determined by persons skilled in the art. Then it is determined whether the agent inhibits or enhances telomere length or maintenance associated with Mre11 complex formation relative to a sample, cells, nuclei or extract not contacted with the agent.

For example, a biological sample, e.g., a physiological sample which comprises cells may be obtained from a mammal, e.g., a mouse or a human. The cells are lysed to yield an extract which comprises cellular proteins. Alternatively, intact cells, e.g., a tissue sample such as paraffin embedded and/or frozen sections of biopsies, are permeabilized in a manner which permits macromolecules, i.e., antibodies, to enter the cell. The antibodies of the invention are then incubated with cells, including permeabilized cells, e.g., prior to flow cytometry, nuclei or the protein extract, e.g., in a Western blot, so as to form a complex. The presence, amount and location of the complex is then determined or detected.

The antibodies of the invention may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the invention. Such solid supports include permeable and semipermeable membranes, glass beads, plastic beads, latex beads, plastic microtiter wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

II. Dosages, Formulations and Routes of Administration of the Agents of the Invention

The agents identified by the methods of the invention may be administered at dosages of at least about 0.001 to about 100 mg/kg, more preferably about 0.01 to about 10 mg/kg, and even more preferably about 0.1 to about 10 mg/kg, of body weight, although other dosages may provide beneficial results. The amount administered will vary depending on various factors including, but not limited to, the agent chosen, the disease or condition, whether prevention or treatment is to be achieved, and if the agent is modified for bioavailability and in vivo stability.

Administration of a sense or antisense nucleic acid molecule may be accomplished through the introduction of cells transformed with an expression cassette comprising the nucleic acid molecule (see, for example, WO 93/02556), or the administration of the nucleic acid molecule itself (see, for example, Felgner et al., U.S. Pat. No. 5,580,859, Pardoll et al., Immunity, 3, 165 (1995); Stevenson et al., Immunol. Rev., 145, 211 (1995); Molling, J. Mol. Med., 75, 242 (1997); Donnelly et al., Ann. N.Y. Acad. Sci., 772, 40 (1995); Yang et al., Mol. Med. Today, 2, 476 (1996); Abdallah et al., Biol. Cell, 85, 1 (1995)), or the nucleic acid molecule introduced into a viral vector or liposomes. Pharmaceutical formulations, dosages and routes of administration for nucleic acids are generally disclosed, for example, in Felgner et al., supra.

Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. When the agents of the invention are employed for prophylactic purposes, agents of the invention are amenable to chronic use, preferably by systemic administration.

One or more suitable unit dosage forms comprising the therapeutic agents of the invention, which, as discussed below, may optionally be formulated for sustained release, can be administered by a variety of routes including oral, or parenteral, including by rectal, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the therapeutic agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and α-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The therapeutic agents of the invention can be delivered via patches for transdermal administration. See U.S. Pat. No. 5,560,922 for examples of patches suitable for transdermal delivery of a therapeutic agent. Patches for transdermal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylene vinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneum, which provides the most resistance to the penetration of molecules. The fabrication of patches for transdermal delivery of therapeutic agents is well known to the art.

For administration to the upper (nasal) or lower respiratory tract by inhalation, the therapeutic agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the therapeutic agent may be administered via nose drops, a liquid spray, such as via a plastic bottle atomizer or metered-dose inhaler. Typical of atomizers are the Mistometer (Wintrop) and the Medihaler (Riker).

The local delivery of the therapeutic agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The formulations and compositions described herein may also contain other ingredients such as antimicrobial agents, or preservatives. Furthermore, the active ingredients may also be used in combination with other therapeutic agents, for example, bronchodilators.

The invention will be further described by, but is not limited to, the following examples.

### EXAMPLE 1

Linkage of Double Strand Break Repair to the Cellular DNA Damage Response Materials and Methods

Cell Lines. HeLa S3 cells were obtained from the University of California-Berkeley Tissue Culture Center. Cell lines derived from patients with Nijmegen breakage syndrome were obtained from K. Sullivan (Children's Hospital of Philadelphia) and P. Concannon (Virginia Mason Research Center, Seattle, Wash.). Ataxia telangiectasia primary fibroblasts (AT3BI) were obtained from J. Murnane (University of California, San Francisco). IMR90, 37Lu, TK6, and K562 cell lines were grown as follows: Cell lines, IMR90 primary diploid fibroblasts were obtained from the American Type Culture Collection and were used at passages 10 to 15, with equivalent results. 37Lu primary fibroblasts were also obtained from the American Type Culture collection and used at passages 3 to 7. 180BR primary fibroblasts were obtained from C. Arlett and were used at passages 8 to 13. GM00637G normal simian virus 40 (SV40)-transformed fibroblasts and the SV40-transformed ataxia-telangiectasia (AT) cell lines GM09607A and GM05849B were obtained from the Cornell Institute for Medical Research. M059J and M059K cells were obtained from M. J. Allalunis-Tumer. Primary fibroblasts were grown in Dulbecco modified Eagle medium with 10% fetal calf serum (FCS), and SV40-transformed fibroblasts were grown in Dulbecco modified Eagle medium with 5% FCS and 5% Cosmic Calf serum (Hyclone, Logan, Utah). K562 CML cells (provided by Peggy Farnham) were grown in RPMI 1640 having 10% Cosmic Calf serum, 1% Pen-Strep and 2 mM L-glutamine. All cells were maintained at 37° C. in a 5% CO₂, 95% air humidified environment. All cells routinely tested negative for mycoplasmas with the MycoTect kit (Gibco, Grand Island, N.Y.). HeLa cells were grown in Joklik's MEM containing 5% newborn calf serum, 50 IU/ml penicillin and 50 μg/ml streptomycin. AT3BI and NBS primary fibroblasts (lines KW and WI 799) were grown in DMEM/10% FCS/5% Fetal Clone III (Hyclone, Logan Utah) and NBS lymphoblasts (lines JS, GM7078, and DST) were grown in RPMI 1640/15% FCS.

Protein Purification. Crude HeLa extract was prepared essentially as described (Nishida et al., 1988). All procedures were carried out at 4° C. A 20% to 50% (NH4)₂SO₄ fraction was loaded on to a DEAE-sephacel column equilibrated in 50 mM Tris-HCl (pH 8.0), 1 mM DTT, 10% glycerol and 50 mM NaCl (TDG+50). The column was eluted with a 50 mM to 500 mM NaCl gradient. hMre11 and hRad50 co-eluted at 180 mM NaCl. Pooled fractions were concentrated by (NH4)₂SO₄ precipitation and resuspended in TDG+200. hMre11 immunoaffinity reagent was constructed by crosslinking affinity-purified hMre11 antiserum with dimethylpimelimidate to protein A-agarose as described (Harlow and Lane, 1988). Pooled DEAE-sephacel fractions were incubated with the α-hMre11 beads in 300 mM NaCl. Beads were washed, and bound proteins quantitatively eluted with Acti-Sep elution media (Sterogene Carlsbad, Calif.). Eluted proteins were dialyzed into 10 mM Tris-8.0, 25 mM NaCl, and fractionated by SDS-PAGE. Proteins were visualized with colloidal Coomassie blue and the unique 95 kDa band was excised for mass spectrometry analysis.

For gel filtration, pooled DEAE-sephacel column fractions derived as above were subjected to gel filtration on Superose 6 FPLC column (Pharmacia, Piscataway, N.J.). Fractions were analyzed by Western blotting with p95, hMre11, and hRad50 antisera.

Mass Spectrometry. Proteins were subjected to in-gel trypsin digestion (Shevchenko et al., 1996). Microelectrospray columns were constructed from 360 micron O.D.×100 micron I.D. fused silica capillary with the column tip tapered to a 5-10 micron opening. The columns were packed with Perceptive Biosystems (Framingham, Mass.) POROS 10 R2, a 10 micron reversed-phase packing material, to a length of 10-12 cm. The flow from the HPLC pumps (typically 150 microliters/minute) was split precolumn to achieve a flow rate of 500 nL/minute. The mobile phase used for gradient elution consisted of (A) 0.5% acetic acid, and (B) acetonitrile/water 80:20 (v/v) containing 0.5% acetic acid. The gradient was linear from 0-40% B in 50 minutes followed by 40-80% B in 10 minutes or 0-60% B in 30 minutes. Mass spectra were recorded on an LCQ ion trap mass spectrometer (Finnigan MAT, San Jose, Calif.) equipped with a microelectrospray ionization source (Gatlin et al., 1998). Electrospray was performed at a voltage of 1.6 kV. Tandem mass spectra were acquired automatically during the entire gradient run as previously described (Link et al., 1997). Tandem mass spectra of peptides from p95 were compared to the protein and gene sequence using the computer program SEQEST (Eng et al., 1994; Yates et al., 1995). Sequences for potential contaminants such as human keratin and bovine trypsin were added to the database.

Two-Hybrid Interaction. hMre11 (nucleotides 160 to 2298 of hMre11 gene encoding the full length nMre11 polypeptide) was expressed as a GAL4 DNA binding domain fusion protein from pASI (Durfee et al., 1993) in the yeast strain, PJ69-4A (James et al., 1996). Following introduction of a human B-lymphoblastoid cell cDNA library into the vector pACT, cDNAs encoding hMre 11 interactors were selected for by growth in the absence of adenine. Apparent adenine prototrophic colonies were retested on plates lacking histidine or adenine, and pACT cDNA clones were isolated from yeast exhibiting adenine and histidine prototrophy and analyzed by DNA sequencing.

Hybridizations. Multiple Tissue Northern Blots (Clontech, Palo Alto, Calif.) were probed as previously described (Dolganov et al., 1996) utilizing a NBS1 or hMRE11 cDNA labeled by random priming as the probe. The Zoo-Blot Southern blot was obtained from Clontech (Palo Alto, Calif.) and probed by standard procedures utilizing the NBS1 cDNA as a probe.

Immunoblotting and immunoprecipitation. p95 antiserum was raised in a rabbit against a fusion protein comprising amino acids 399-751 of human p95 fused to glutathione-S-transferase (GST). Affinity purification of anti-p95 antiserum was performed (Dolganov et al., 1996) over GST (to remove GST reactivity from the antiserum) and GST-p95 columns constructed with actigel resin (Sterogene, Carlsbad, Calif.). The hMre11 monoclonal antibody was derived from a mouse immunized with a 6X-His-hMre11 fusion protein (the fusion protein included the complete hMre11 polypeptide) in the University of Wisconsin Hybridoma facility.

Whole cell extracts (from 3×10⁵ cells) were prepared (Dolganov et al., 1996) and fractionated in 7.5% SDS-PAGE gels. Proteins were transferred to nitrocellulose and immunoblots were performed (Dolganov et al., 1996) with p95, hMre11, and hRad50 antisera on the same filter in succession. Immunoprecipitations were performed on K562 lysates with p95, hMre11, hRad50 or the respective pre-immune antisera (Dolganov et al., 1996). Immunoprecipitates were fractionated, transferred to nitrocellulose and immunoblotted as above.

Chromosomal localization of p95. Metaphase chromosomes were prepared from phytohemagglutin-stimulated peripheral blood lymphocytes from a normal human subject. The NBS1 probes (clones 926991 and 1083839) were biotin-labeled by nick translation using Bio-16-dUTP (Enzo Diagnostics, Farmingdale, N.Y.) and fluorescence in situ hybridization was performed (Rowley et al., 1990). Hybridization was detected with fluorescein-conjugated avidin (Vector Laboratories, Burlingame, Calif.), and chromosomes were identified by staining with 4,6-diamidino-2-phenylindole-dihydrochloride (DAPI).

Immunofluorescence. Primary fibroblasts were grown on glass slides, irradiated and fixed. For double immunolabeling, cells were fixed in 3.5% paraformaldehyde and permeabilized (Scully et al., 1997). Cells were incubated with affinity-purified rabbit p95 antiserum (as above) and a 1:50 dilution of anti-hMre11 monoclonal ascites (line 8F3) for 1 hour at room temperature. After washing in PBS, cells were incubated with FITC-conjugated goat anti-rabbit and Texas Red-conjugated donkey anti-mouse antisera (Jackson Immunoresearch, West Grove, Pa.) for 1 hour at room temperature. Cells were then washed, counterstained with DAPI, and mounted.

In IRIF assays, a minimum of 200 nuclei were analyzed for each cell line, treatment, and antibody examined. Unirradiated samples were fixed and processed as above along with irradiated samples. Mutant NBS cells and normal controls were processed simultaneously and treated identically.

Results

Purification of the hMre11/hRad50 Complex from HeLa Extract

The 95 kDa and 200 kDa components of the hMre11/hRad50 protein complex were purified from a HeLa cell extract for direct protein sequencing. A 20% to 50% (NH4)₂SO₄ precipitate was separated by anion-exchange chromatography, and fractions containing hRad50 and hMre11 were identified by Western blotting. The hRad50/hMre11 complex was further purified from peak fractions by immunoaffinity chromatography and fractionated by SDS-PAGE. The hMre11/hRad50 protein complex includes five proteins of 81 kDa, 95 kDa, 150 kDa, 200 kDa, and approximately 400 kDa, which are immunoprecipitable by hMre11 antiserum (Dolganov et al., 1996). As expected, these proteins were readily visible on a Coomassie-stained gel. The 95 kDa and 200 kDa bands (referred to henceforth as p95 and p200) were excised from the gel and subjected to mass spectroscopic analysis.

Mass Spectrometry of Purified Proteins

The p95 protein was digested with trypsin and analyzed by LC/MS/MS to acquire tandem mass spectra for sequence analysis. A tandem mass spectrum for a peptide of molecular weight 1,420.2 Da was interpreted to represent the sequence NPSGLNDDYGQLK (SEQ ID NO: 1). A tBLASTx search of dbEST found a match to the sequence NPSGLNDDYGQLK (SEQ ID NO: 1) in human EST 926991.

A similar analysis of the p200 band identified this protein as fatty-acid synthase (FAS) (Jayakumar et al., 1996). Since subsequent gel filtration chromatography indicated that FAS did not co-elute with the hRad50/hMre11 complex, it was concluded that the presence of FAS among the immunoaffinity purified proteins was an artifact of the isolation procedure.

Cloning of the NBS1 cDNA

The EST cDNA clone encoding p95 peptides (Table 1) was obtained from the IMAGE consortium (clone identification number 926991 [EST11]. This clone, and a second overlapping human EST, 1083839 [EST30], were sequenced in their entirety. The combined DNA sequence spanned 4,483 bp and contained a 2,265 bp open reading frame, sufficient to encode a protein with a predicted molecular weight of 85 kDa. The resulting cDNA has been designated NBS1 (for Nijmegen breakage syndrome). Comparison of the open reading frame with tandem mass spectra obtained from purified p95 identified 16 additional peptide matches (Table 1). The predicted protein has no homology to any known proteins. Contrary to expectations, the predicted p95 protein is essentially unrelated to the S. cerevisiae Xrs2 protein; the two proteins share only modest homology (28% identity) over the N-terminal 115 amino acids.

TABLE 1

| | | |
|---|---|---|
| Peptide^{a} | SEQ ID NO: | Position^{b} |

| | | |
|---|---|---|
| -QPPQIESFYPPLDEPSIGSK- | 2 | 189-209 |
| -LSSAVVFGGGEAR- | 3 | 238-251 |
| -WIQSIMDMLQR- | 4 | 289-299 |
| -QGLRPIPEAEIGLAVIFMTTK- | 5 | 300-320 |
| -TTTPGPSLSQGVSVDEK- | 6 | 335-351 |
| -MLSQDAPTVKE- | 7 | 395-404 |
| -TSSNNNSMVSNTLAK- | 8 | 409-423 |
| -IPNYQLSPTKLPSINK- | 9 | 426-441 |
| -NYFQPSTKK- | 10 | 458465 |
| -NKEQHLSENEPVDTNSDNNLFTDTDLK- | 11 | 503-529 |
| -EMDDVAIEDEVLEQLFK- | 12 | 552-558 |
| -MDIETNDTFSDEAVPESSK- | 13 | 595-613 |
| -ELKEDSWAK- | 14 | 625-635 |
| -KLLLTEFR- | 15 | 653-660 |
| -NPSGINDDYGQLK-^{c} | 16 | 671-683 |
| -EESLADDLFR- | 17 | 736-745 |

^{a}Mass spectrometry is unable to distinguish L/I and Q/K. The indication of L, I, Q, and K residues in the peptide sequences is based on the p95 ORF.
^{b}Numbering system is based on the ORF of the p95 cDNA.
^{c}This peptide was sequenced by tandem mass spectrometry and used for EST database searches. The remaining peptides corresponded to the derived amino acid sequence from the p95 ORF.

Two-Hybrid Interaction of p95 and hMre11

In parallel with the approach described above, a two hybrid interaction screening was employed to identify hMre11-interacting proteins. The hMRE11 cDNA was cloned into the vector pAS1 as an in-frame fusion with the Gal 4 DNA binding domain (Durfee et al., 1993), and cDNAs encoding interacting proteins were isolated from a human B lymphoblastoid cDNA library by two hybrid screening. DNA sequence and hybridization analyses revealed that twenty independent NBS1 cDNA clones were among the interactors. The largest NBS1 cDNA obtained in this screen began at amino acid position 363 of the p95 protein.

Expression of NBS1

Northern blot analysis with an NBS1 cDNA probe revealed two NBS1 mRNAs, a 4.4 kb transcript that was relatively abundant in all tissues, and a 2.6 kb transcript that was present at high levels in testis. The 4.4 kb mRNA, and not the 2.6 kb mRNA, was detected with a probe from the 3′ non-coding segment of the NBS1 cDNA, indicating that the two transcripts arise from the same locus, but differ in the amount of 3′ untranslated sequence that they contain. The same Northern blot filter was hybridized to an hMRE11 cDNA probe. Two hMRE11 mRNA species were detected; a 6.6 kb mRNA that was present in all tissues, and a 2.4 kb mRNA that was most abundant in testis. This pattern of expression is analogous to that of the murine MRE11 gene (Petrini et al., 1995). The expression patterns of the NBS1 2.6 kb MRNA and the 2.4 kb hMRE1 mRNA were identical, consistent with the observation that their respective protein products function together in the same complex.

Interaction of p95 with the hRad50/hMre11 Complex

To confirm the results of the immunoaffinity purification, a series of immunoprecipitations was performed using K562 cell extracts with hMre11, hRad50, and p95 antisera (Dolganov et al., 1996). Immunoprecipitates were subsequently analyzed by Western blotting with the same antisera. All three proteins (hRad50, hMre11, and p95) were precipitated with the three respective antisera but not with the corresponding pre-immune sera.

The association of these three proteins was also confirmed by gel filtration chromatography. hMre11/hRad50-containing fractions from the DEAE-sephacel column described above were pooled and separated on a Superose 6 gel filtration column. Western blotting of fractions from the sizing column with hMre11, hRad50, and p95 antisera was carried out. The three proteins co-chromatographed in a single peak corresponding to a molecular weight of approximately 1,600 kDa. hMre11, hRad50, or p95 proteins were not detected in later column fractions corresponding to lower molecular weight species. This observation suggests that the vast majority of hMre11, hRad50, and p95 in the cell are present in the high molecular weight complex.

Conservation of p95

Human metaphase cells were used for fluorescence in situ hybridization (FISH) with the NBS1 cDNA. Co-hybridization of probes EST11 and EST30 resulted in specific labeling only of chromosome 8. Specific labeling of 8q21.2-22.1 was observed on four (2 cells), three (5 cells), two (12 cells), or one (6 cells) chromatid(s) of the chromosome 8 homologues in 25 cells examined. Of 61 signals observed, 53 (87%) were located at 8q21.2-22.1. Of these, 4 (7.5%) signals were located at 8q21.2, 46 (87%) signals were located at 8q21.3, and 3 (5.5%) signals were located at 8q22.1. Eight background signals were observed at other chromosomal sites. Six of these were single signals, and none of these chromosomal bands were labeled more than once. Doublet signals were observed once at 5q14. A specific signal was observed at 8q21.3 in an additional hybridization experiment using this probe. These results suggest that the gene coding for p95 is localized to chromosome 8, band q21.3. Previous studies have demonstrated that the gene defective in the chromosome instability syndrome, Nijmegen Breakage Syndrome (NBS), maps to this locus (Matsuura et al., 1997; Saar et al., 1997).

To determine whether p95 protein was present in cell lines established from NBS patients, extracts were prepared from cell lines from five patients and subjected to Western blot analysis with p95 antiserum. p95 was not detected in any of the patient samples examined, although the levels of hMre11 and hRad50 were normal. These data suggest that NBS is attributable to deficiency in the gene encoding p95.

To assess whether hRad50 and hMre11 interact in the absence of p95, immunoprecipitations with anti-hMre11 antibody were carried out using crude extracts from a representative NBS cell line (JS). It was found that hMre11 and hRad50 were co-immunoprecipitated by anti-hMre11 antibody from NBS extracts and control TK6 extracts. The hMre11/hRad5 interaction is unaffected by the absence of p95, indicating that these two proteins interact directly. Radiation Induced Foci In human cells, hMre11 and hRad50 colocalize in large nuclear foci following treatment with agents that induce DSBs. To examine whether p95 is also present in ionizing radiation induced foci (IRIF), 37Lu (normal diploid fibroblasts) were plated on glass slides, irradiated at a does of 12 Gy, and doubly strained with hMre11 and p95 antisera at 8 hours post-irradiation. hMre11 and p95 IRIF colocalized in irradiated cells, whereas diffuse nuclear staining with both antisera was observed in unirradiated control cells. As expected from Western blotting, p95 was not detectable in NBS cell lines by immunofluorescence. This observation is consistent with the notion that hMre11, hRad50, and p95 function in the same protein complex during the normal cellular response to DNA damage.

Normal DNA damage responses such as cell cycle arrest, inhibition of DNA synthesis, and the induction of p53 are abrogated in NBS cells (Jongmans et al., 1997; Sullivan et al., 1997). Therefore, to determine whether the hMre11 IRIF response was intact in cell lines established from NBS patients, NBS fibroblasts (W1799 and KW) and normal control fibroblasts (IMR90 and 37Lu) were plated on slides as above, irradiated, and stained with hMre11 or p95 antiserum. About p95 IRIF responses of normal cells were consistent with previous studies 64% to 85% of irradiated cells were positive for IRIF at 8 hours post-irradiation. However, immunofluorescence analysis of over 1500 NBS cells showed that, in contrast to normal cells, the intranuclear levels of hMre11 and hRad50 were drastically reduced n both NBS cell lines, irrespective of prior irradiation. Thus, hMre11-hRad50 IRIF do not form in the absence of p95.

Discussion

Nijmegen Breakage Syndrome is an autosomal recessive disorder characterized by developmental abnormalities, variable immune deficiency, and marked predisposition to malignancy. Cells established from NBS patients are sensitive to IR, exhibit chromosome fragility, and fail to activate cell cycle checkpoints in response to DNA damage (reviewed in Shiloh, 1997; van der Burgt et al., 1996; and Weemaes et al., 1994). With respect to these features, the mutations that result in NBS and AT have essentially identical phenotypic outcomes. This suggests that the NBS1 and ATM gene products effect the same or closely related functions in the cellular DNA damage response (Shiloh, 1997).

As shown above, deficiency in a member of the hMre11/hRad50 protein complex, p95, is the cause of NBS. The evidence for this is: 1) the p95 locus maps to 8q21.3, the region to which the NBS locus was previously localized (Matsuura et al., 1997; Saar et al., 1997); 2) the NBS1 and p95 cDNA sequences are identical; and 3) the p95 protein is absent from extracts of NBS cell lines that harbor NBS1 mutations. In light of the defects associated with NBS, these findings indicate that the hMre11/hRad50 protein complex is intimately involved in initiating the cellular DNA damage response.

The hMre11/HRad50 protein complex may function as a sensor of DNA damage

The hMre11/hRad50 protein complex forms discrete nuclear foci (RIF) following the induction of DSBs by ionizing radiation in normal cells. In contrast, IRIF do not form in NBS cells, suggesting that p95 is required for the relocalization of the hMre11/hRad50 protein complex to DSBs. This observation raises the possibility that p95 regulates the hMre11/hRad50 protein complex by transducing a signal originating from the site(s) of DNA damage. In normal cells, this signal leads to relocalization of the complex, whereas the signal is not transduced in NBS cells and so movement of the complex does not occur. A similar, though less severe defect was observed in SV40 transformed AT cell lines (Shiloh, 1997). These observations are consistent with the hypothesis that mutations in NBS and AT affect proximate functions in the DNA damage response.

However, the defect revealed by this aberrant IRIF response cannot account for the absence of DNA damage dependent cell cycle checkpoints and diminished p53 responses observed in NBS cells (Jongmans et al., 1997; Shiloh, 1997; Sullivan et al., 1997). These aspects of the NBS phenotype indicate that p95, and by extension, the hMre11/hRad50 protein complex, are an integral part of the signal that activates the cellular DNA damage response. In this regard, recent cytologic analyses of the hMre11/hRad50 protein complex in normal human cells are significant. DNA damage was induced in discrete subnuclear volumes, and DNA repair at those sites was monitored. hMre11 associated with DSBs within thirty minutes of their induction, and uniform distribution of the protein was restored upon subsequent DSB repair. The observed behavior of hMre11 at the sites of DNA damage is consistent with the notion that the hMre11/hRad50 protein complex functions as a DNA damage sensor, and readily accounts for the DNA damage-dependent cell cycle checkpoint defects associated with NBS. The DNA repair functions of the hMre11/hRad50 protein complex are thus physically associated with the activation of other aspects of the cellular DNA damage response.

The hMre11/hRad50 Protein Complex in NBS Cells

The physical association of hMre11 and hRad50 is unaffected by the absence of p95. Similarly, the association of ScMre11 and ScRad50 does not appear to depend upon ScXrs2 (Johzuka and Ogawa, 1995; Ogawa et al., 1995). However, cytologic analyses reveal that the disposition of hMre11 and hRad50 is abnormal in NBS cells. The uniform nuclear distribution typical of hMre11 and hRad50 in unirradiated normal cells is not seen in NBS cells. Instead, the intranuclear abundance of hMre11/hRad50 appears to be reduced with a concomitant increase in the cytoplasmic level. Western blotting of fractionated NBS cell extracts shows that the protein is readily detectable in the nuclear fraction, though its level is somewhat diminished. This is expected, since in murine embryonic stem cells, muMre11 is required for viability (Xiao and Weaver, 1997). Whereas loss of p95 may decrease the intranuclear abundance of hMre11 and hRad50, the levels of these proteins must be sufficiently high to support cell viability.

Divergence of the hRad50/hMre11 Protein Complex

hRad50 and hMre11 are human homologues of the E. coli proteins SbcC and SbcD (Sharples and Leach, 1995). SbcC and SbcD function in a protein complex, SbcCD, that possesses ATP-dependent double-stranded exonuclease activity and ATP independent single-strand endonuclease activity (Connelly et al., 1997; Connelly and Leach, 1996; and Gibson et al., 1992). Genetic evidence from S. cerevisiae suggests that the Mre11/Rad50/Xrs2 protein complex functions as a nuclease in that organism (Ivanov et al., 1996; Ivanov et al., 1994; and Tsubouchi and Ogawa, 1998). The biochemical activities of the hMre11/hRad50 protein complex have not been established, but the conservation of hMre11 and hRad50 suggests that they also encode nuclease activity.

It was found that p95 was not the human Xrs2 homologue. The replacement of Xrs2 by p95 in humans may indicate that the function(s) mediated by these proteins are not conserved. This seems an unlikely possibility in light of the conservation of Mre11 and Rad50, particularly since neither Xrs2 nor p95 appears to function outside of its respective complex (Petrini et al., 1997). An alternative interpretation based on the NBS phenotype is that Xrs2 and p95 link the conserved activities of Mre11/Rad50 to the cellular DNA damage response in their respective organisms. In this sense, these divergent proteins could be considered functional analogues. The lack of similarity between p95 and Xrs2 would therefore reflect those features of the DNA damage response, and the roles of the Mre11/Rad50 nuclease within it, that are unique to each organism.

Phenotypic Similarity of S. cerevisiae and Human Mutants

The phenotypic features of S. cerevisiae mre11/rad50/xrs2 mutants are reminiscent of those observed in a number of human chromosomal instability syndromes such as NBS, AT, and Bloom syndrome (Bigbee et al., 1989; Bubley and Schnipper, 1987; Chaganti et al., 1974; Hojo et al., 1995; Langlois et al., 1989; Meyn, 1993). The chromosome fragility of NBS cells demonstrates that defects in the hMre11/hRad50 protein complex can result in similar phenotypic outcomes in mutant human cells.

Analogy of the S. cerevisiae and human phenotypes might also include DNA recombination defects, raising the possibility that immune dysfunction in NBS patients is attributable to DNA recombination defects. This interpretation is supported by the preponderance of chromosomal rearrangements involving chromosomes 7 and 14 in peripheral blood lymphocytes (reviewed in Weemaes et al., 1994). If the hMre11/hRad50 protein complex mediates single-strand DNA endonuclease activity similar to that of SbcCD (Connelly and Leach, 1996), the complex might be important for the resolution of hairpin intermediates generated in the V(D)J recombination process (Gellert, 1997).

In this regard, it is noteworthy that similar fragility of chromosomes 7 and 14 is seen in AT patients, yet the immune defects observed in that disease do not appear to result from defects in V(D)J recombination (Hsieh et al., 1993). Immunoglobulin heavy chain rearrangements in the NBS lymphoblastoid cell line, GM7078, have been analyzed by DNA sequencing and found to be normal (Petrini et al., 1994). However, quantitative analysis of V(D)J recombination in NBS cells is required to adequately address the role of the hMre11/hRad50 protein complex in this process. Lymphocyte-specific recombination is also required for immunoglobulin class switching (reviewed in Stavnezer, 1996), and it is conceivable that this process is also affected in NBS patients. Furthermore, DNA recombination in other contexts, such as in meiotic recombination, may also be affected by mutation of NBS1.

p95 Links Recombinational DNA Repair to Cancer Predisposition

Genomic instability is frequently observed in human cancer predisposition syndromes (Cleaver, 1989; German, 1983; Jackson, 1995; Kolodner, 1995; and Timme and Moses, 1988). Among such syndromes are congenitally acquired deficiencies in nucleotide excision repair and DNA mismatch repair. Since chromosomal rearrangements and changes in chromosome number are common features of malignant cells, errors in recombinational DNA repair are likely to play an important role in neoplasia (reviewed in Rabbitts, 1994; and Rowley, 1994). The implication of p95 and the hMre11/hRad50 protein complex in NBS constitutes an important link between congenital recombinational DNA repair deficiency and genomic instability associated with the predisposition to malignancy.

### EXAMPLE 2

### Cell Cycle Regulated Association of Rad50/Mre11/Nbs1 with TRF2 and Human Telomeres

Methods

Protein extracts. Nuclear and whole cell extracts were prepared as described previously (van Steensel et al., 1998; Dignam et al., 1983; Zhang et al., 1992). Briefly, nuclear extracts were prepared on ice by extraction of nuclei with 0.42 M KCl in buffer C (20 mM Hepes-KOH, pH 7.9, 25% glycerol, 0.1 mM EDTA, 5 mM MgCl₂, 1 mM Dithiothreitol (DTT), aprotinin, leupeptin, and pepstatin (each at 1 μg/ml), and 0.5 mM phenylmethysulfonyl fluoride (PMSF)). Whole cell extracts were prepared by resuspending cell pellets in buffer C containing 0.42 M KCl and 0.2% Nonidet P-40. The extracts were then dialyzed at 4° C. overnight against 100 mM KCl in buffer D (20 mM Hepes-KOH, pH 7.9, 20% glycerol, 0.2 mM EDTA, 0.2 mM EGTA, 1 mM DTT, and 0.5 mM PMSF) and stored at −80° C. Protein concentrations were determined using the Bradford assays (BioRad, Hercules, Calif.) with BSA as a standard.

Isolation of TRF2 complexes from HeLa nuclear extracts. For large scale immunoprecipitation, nuclear extracts from HeLa S3 cells (610 mg) were loaded on to a 5-ml Heparin column (Pharmacia Biotech) equilibrated in buffer D with 100 mM KCl and eluted with a 0.2 M-1 M KCl linear gradient. TRF2-containing fractions (from 0.5 M-1M KCl) were pooled and dialyzed against 100 mM KCl in buffer D. TRF2 immunoaffinity beads were prepared by cross-linking 68 μg of Ab#647 to 3.5 ml protein A beads (Pharmacia Biotech) using dimethylpimelimidate as described in Harlow et al. (1988). The cross-linked anti-TRF2 beads were washed with 0.1 M glycine (pH 2.5), equilibrated with PBS, and subsequently incubated with pooled TRF2-containing heparin column fractions in buffer D with 100 mM KCl and 0.1% Nonidet P-40. Beads containing TRF2 immuno-complex were washed 5 times in buffer D containing 300 mM KCl, 0.2% Nonidet P-40 and bound proteins were eluted with 0.1M glycine (pH 2.5). The eluted proteins were precipitated by 20% trichloacetic acid and 0.015% deoxycholate and fractionated on SDS-PAGE. Proteins were visualized with Coomassie blue and bands corresponding to 150 kDa, 70 kDa (doublet) and 60 kDa were excised for mass spectrometric analysis.

Mass spectrometry. Proteins were digested in gel with trypsin (Roche Diagnostics) as described in Sherchenko et al. (1996) and the resulting peptides were analyzed by nanoelectrospray tandem mass spectrometry (Wilm et al., 1996) on a prototype quadrupole time-of-flight tandem mass spectrometer (Sherchenko et al., 1997) (PE Sciex). Peptide sequence tags (Mann et al., 1994) were constructed from the tandem mass spectra and searched against a non-redundant protein database (NRDB) maintained and updated regularly at the European BioInformatics Institute (Hinxton, UK) using the program PeptideSearch developed in M. Mann's group at the EMBL in Heidelberg. Peptide sequences of the retrieved proteins were verified by matching the calculated fragment ion masses to signals observed in the respective tandem mass spectra.

Transfection and Infection. HeLa cells were transfected transiently with either a Myc-tagged pCDNA3-TRF2^{ΔBΔM} or with pTetNFlag-TRF2^{ΔBΔM} in conjunction with pUHD15-1 (van Steensel et al., 1998; van Steensel et al., 1997). As a control, transfection was also carried out with pCDNA3 vector or with the pTetNFlag vector in conjunction with pUHD15-1 plasmid (van Steensel et al., 1998; van Steensel et al., 1997). Infection of HeLa cells was performed with either retrovirus pLPCNMyc-TRF2^{ΔBΔM} or pLPCNMyc vector (A. Smogorzewska and T. de Lange, unpublished). Cells were extracted with Triton-X-100 and fixed 24 hours post-transfection or post-infection without drug selection.

Immunoblotting and immunoprecipitation. TRF2 Ab#647 was raised in a rabbit against purified [His]6-tagged full-length TRF2 expressed from baculovirus (bacTRF2). Mouse anti-TRF1 and mouse anti-TRF2 antisera were raised in mice against full-length TRF1 and TRF2 expressed from baculovirus. Antiserum #765 to RAP1 is described elsewhere (Li et al., 2000). Ab#647 was affinity purified against bacTRF2 coupled to CNBr-activated agarose using standard procedures (Harlow et al., 1988). Immunoblotting was carried out essentially as described in van Steensel et al. (1998) with HeLa nuclear extract (20 μg) or whole-cell extract (40 μg) fractionated on 8% SDS-PAGE and transferred to nitrocellulose. Immunoblots were performed with monoclonal M2 anti-FLAG antibody (Sigma), mouse anti-TRF1 antibody, or with rabbit antiserum against Rad50 (Dolganov et al., 1996), Mre11 (Dolganov et al., 1996), Nbs1 (Carney et al., 1998), TRF1 (van Steensel et al., 1997) (Ab#371), TRF2 (Ab#647, above), or RAPI (Ab#765, above). Immunoprecipitation was performed with whole cell extract (7 mg) and TRF2 preimmune serum, Ab#647 or M2 anti-FLAG antibody. Immunoprecipitates were fractionated on 8% SDS-PAGE, transferred to nitrocellulose, and immunoblotted as above.

Immunofluorescence. Cells grown on coverslips were rinsed with PBS and then were either fixed immediately or incubated in Triton X-100 buffer (0.5% Triton X-100, 20 mM Hepes-KOH, pH 7.9, 50 mM NaCl, 3 mM MgCl₂, 300 mM sucrose) at room temperature for 5 minutes prior to fixation (Scully et al., 1997). Fixation was carried out in PBS-buffered 3% paraformaldehyde and 2% sucrose at room temperature for 10 minutes, followed by permeabilization in Triton X-100 buffer at room temperature for 10 minutes. For dual immunostaining, cells were blocked with 0.5% BSA (Sigma) and 0.2% gelatin (Sigma) in PBS and then incubated at room temperature for 2 hours with either mouse anti-TRF1 (1:5000) or mouse anti-TRF2 (1:1500) in conjunction with affinity-purified rabbit antiserum (1:150) against Rad50, Mre11, and Nbs1 (see above). Following incubation, cells were washed in PBS and incubated with fluorescein isothiocyanate (FITC)-conjugated donkey anti-rabbit and tetramethyl rhodamine isothiocyanate (TRITC)-conjugated donkey anti-mouse antibodies (1:100 dilution; Jackson Laboratories) at room temperature for 1 hour. Cells were then washed and DNA was stained with 4, 6-diamidino-2-phenylindole (DAPI; 0.2 μg/ml). Images were recorded on a Zeiss Axioplan microscope with either a Photometrics charge-coupled device camera or with a Hammamatsu C4742-95 camera, processed in either IP Lab or in Open Lab and merged in Adobe Photoshop.

Cell cycle analysis and gamma irradiation. Synchronization of HeLa1.2.11 cells was essentially done as described in Bischoffet al. (1998). Exponentially growing HeLa1.2.11 cells were arrested in growth media with 2 mM thymidine for 14 hours followed by washing in PBS (3 times) and release into fresh medium for 11 hours. Cells were then arrested a second time by addition of 1 μg/ml aphidicolin to the media for 14 hours and then washed in PBS (3 times) before release into fresh medium for 0-16 hours. For FACS analysis, one million cells of asynchronous HeLa and synchronized HeLa cells that were released for 0-10 hours were collected by trypsinization, and resuspended in PBS and 2 mM EDTA. Cells were fixed in 70% ethanol, digested with RNase A (0.02 μg/μl), stained with 50 μg/ml propidium iodide, and analyzed using a Becton-Dickinson FacsScan and CellQuest software.

For γ-irradiation, cells were irradiated in a ¹³⁷CS source at a dose of 12 Gy and harvested at the indicated time points.

Results

Telomeres allow cells to distinguish natural chromosome ends from damaged DNA and protect the ends from degradation and fusion. In human cells, telomere protection critically depends on the TTAGGG repeat binding factor, TRF2 (Broccoli et al., 1997; Bilaud et al., 1997; van Steene et al., 1998; Karlseder et al., 1999; and Smogorzewska et al., 2000), which has been proposed to remodel telomeres into large duplex loops (t-loops).

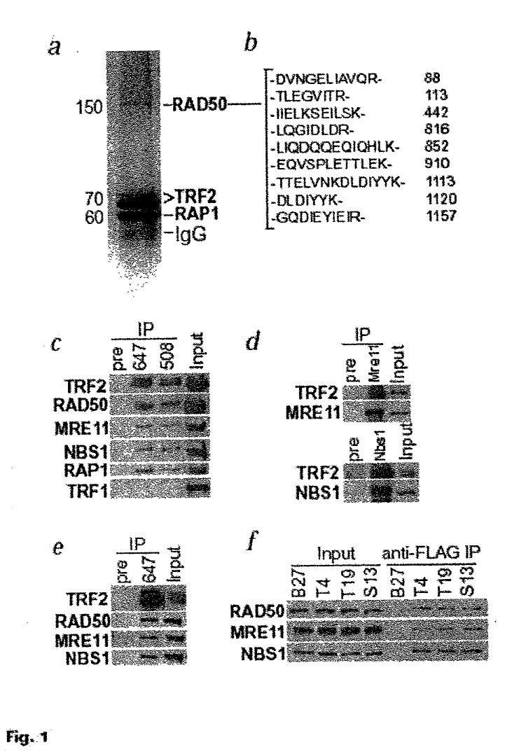

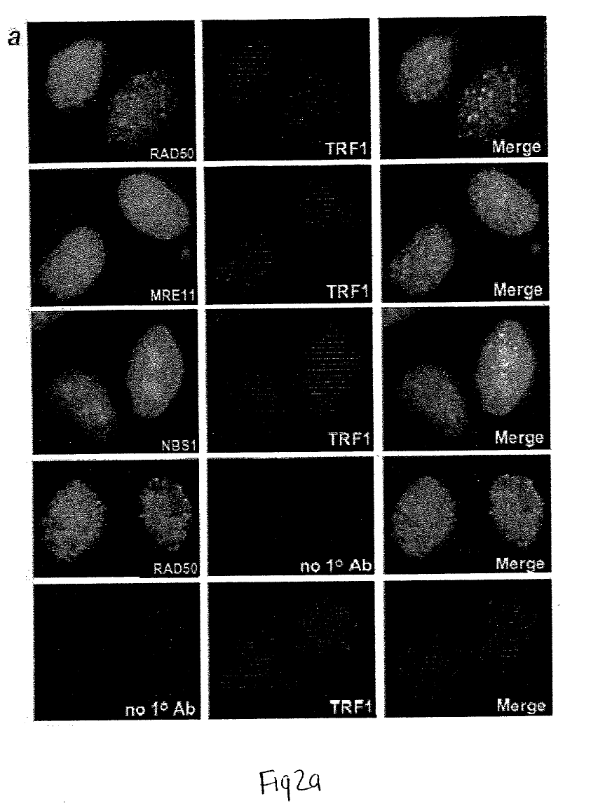

TRF2 complex was isolated by immunoprecipitation from Heparin-Sepharose fractionated HeLa nuclear extract and was found to contain polypeptides migrating at 150 kDa, 70 kDa, and 60 kDa (FIG. 1A). Since these bands were consistently observed in TRF2 immunoprecipitates but not in control experiments with pre-immune serum, their identity was examined by nanoelectrospray tandem mass spectrometry of tryptic peptides (Wilm et al., 1996). The sequence of peptides from the 60 kDa band revealed hRap1, the human ortholog of budding yeast Rap1p which was recently identified as a TRF2-interacting factor in a yeast 2-hybrid screen (Li et al., 2000). The 70 kDa doublet contained TRF2. Human Rap1 and TRF2 were recovered at apparent 1:1 stoichiometry (FIG. 1A).

The sequence of nine peptides from the 150 kDa band identified Rad50 (FIG. 1B). Rad50 is stably associated with Mre11 and Nbs1 (Dolganov et al., 1996; Luo et al., 1999) in a complex that functions in the response to DSBs in both yeast and mammals (Petrini, 1999). The human Mre11 complex functions in DNA damage detection and in the subsequent activation of the DNA damage-dependent S-phase checkpoint (Stewart et al., 1999; Nelms et al., 1998; Carney et al., 1998), whereas studies in Saccharomyces cerevisiae reveal a key function in recombinational DNA repair and telomere maintenance (Bressan et al., 1999; Haber, 1998; Usui et al., 1998; Nugent et al., 1998; Moreau et al., 1999). Given the conservation of Mre11 and Rad50, it is likely that the mammalian Mre11 complex also functions in recombinational DNA repair. In agreement, the mouse Mre11 and Rad50 genes are essential (Luo et al., 1999; Xiao et al., 1997).

TRF2 immunoprecipitates collected with two unrelated TRF2 sera (#508 and #647) contained Rad50, Mre11, Nbs1 as well as hRap1 (FIG. 1). Other telomeric proteins, including TRF1 and tankyrase, were not recovered with TRF2. Immunoprecipitation of Mre11 or Nbs1 brought down TRF2 (FIG. 1D), confirming the association of the Mre11 complex with TRF2. The Mre11 complex was also found in TRF2 immunoprecipitates from primary human fibroblasts (FIG. 1E). The fraction of the Mre11 complex associated with TRF2 is relatively small (1-5% of HeLa Rad50 was recovered with TRF2; data not shown) and vice versa, only a minor fraction of TRF2 appears to be associated with Rad50 (FIG. 1A). Other abundant nuclear proteins did not interact with TRF2 (e.g., GCN5 and XPA, data not shown), indicating that the association of TRF2 with the Mre11 complex is not simply due to its abundance.

The association of TRF2 with Rad50/Mre11/Nbs1 was mediated by DNA tethering in cell lines T4 and T19 that express a FLAG-tagged version of TRF2, TRF2^{ΔBΔM}, that does not bind to DNA. Cell line S13, expressing a FLAG-tagged, DNA binding competent version of TRF2 (TRF₂^{ΔB}), was examined in parallel. The Mre11 complex was present in FLAG immunoprecipitates from S13 as well as T4 and T19, but not in the B27 negative (empty vector) control (FIG. 1F) showing that recovery of Mre11 in TRF2 immunocomplex is not dependent on the DNA binding activity of TRF2. Moreover, high salt conditions (500 mM KCl), which dissociated most of the TRF2 from telomeric DNA, did not disrupt the interaction of TRF2 with the Mre11 complex, nor does the Mre11 complex appear in TRF1 immunoprecipitates (data not shown). Together, these data support a bonafide interaction between the Mre11 complex and TRF2, and rule out DNA tethering as the basis for their coimmunoprecipitation.

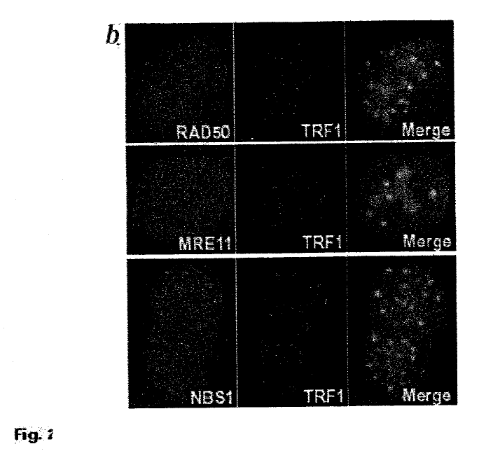

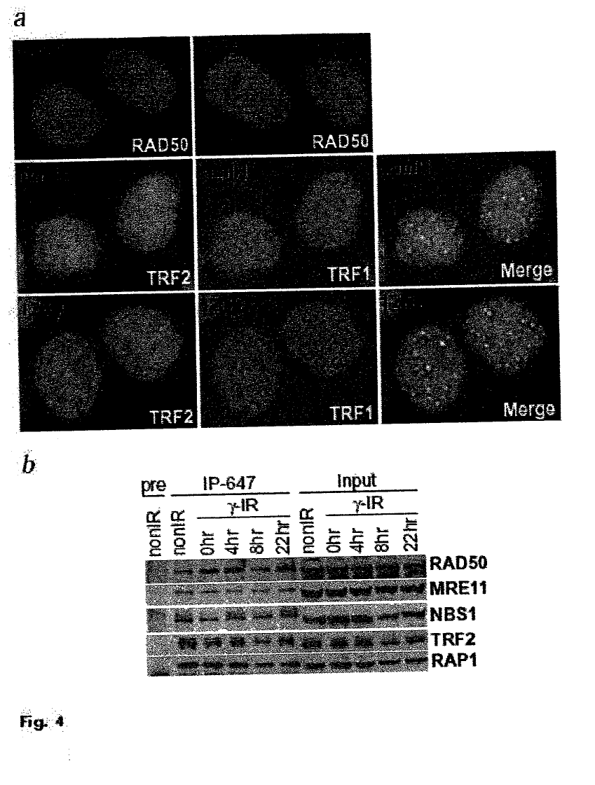

To examine the presence of Rad50/Mre11/Nbs1 at telomeres, dual indirect immunofluorescence (IF) with TRF1 was used, which was previously shown to be a specific marker for interphase telomeres (van Stensel et al., 1997; Cheng et al., 1995). Because the Mre11 complex shows a diffuse overall nuclear signal (Carney et al., 1998; Welms et al., 1998; Maser et al., 1997) which could mask its presence at telomeres, IF studies were performed after extraction of nucleoplasmic proteins with detergent (Triton X-100). Detergent extraction did not affect the distribution of TRF1, TRF2, and hRap1 (data not shown). Unlike the bright overall staining of unextracted cells (see FIG. 4A), Rad50 antibody revealed a punctuate pattern in detergent-extracted interphase nuclei (FIG. 2A) suggesting that a small fraction of Rad50 located to specific subnuclear sites. This pattern was distinct from the radiation-induced foci which are larger and less homogeneous in size (Carney et al., 1998; Maser et al., 1997) (see FIG. 4A). Many of the sites of Rad50 staining were found to co-localize with TRF1, although non-telomeric Rad50 was also observed and it was not established whether each telomere contains Rad50. Mre11 generally displayed a more homogenous nuclear pattern, but, nevertheless, showed a number of dots in each nucleus that co-localized with TRF1, consistent with the presence of Rad50 and Mre11 at telomeres (FIG. 2). Similar co-localization could be observed in dual IF with Rad50 and TRF2 (data not shown).

Co-localization of Rad50 and Mre11 with TRF1 and TRF2 was also observed in two human ALT cell lines that maintain telomeric DNA using a telomerase-independent pathway (Bryan et al., 1997). Unlike HeLa cells, which express telomerase and maintain telomeres of 6-20 kb, the ALT cell lines W138VA13/2Ra and GM847 have extremely long and heterogenously-sized telomeres resulting in much stronger TRF1 and TRF2 signals (Yeager et al., 1999) (FIG. 2B). The telomeric DNA in ALT cells has been shown to co-localize with several proteins involved in DNA synthesis and repair as well as the PML protein (referred to as ALT-associated PML bodies) (Yeager et al., 1999). Confirming the results with HeLa cells, the two ALT cell lines showed co-localization of Rad50 and Mre11 with TRF1 (FIG. 2B).

To assess whether the telomeric localization of Rad50 was mediated by TRF2, HeLa cells were examined that transiently express the dominant negative allele of TRF2 (TRF₂^{ΔBΔM}) which inhibits the accumulation of the endogenous TRF2 on telomeres (van Steensel et al., 1998). Dual IF for Rad50 and TRF1 was used to determine the percentage of nuclei lacking Rad50 at telomeres in cells with normal TRF2 function versus cells expressing TRF₂^{ΔBΔM} (Table 2). In control cells, the fraction of nuclei lacking telomeric Rad50 ranged from 3.6 to 5.7%. By contrast, in cultures that expressed TRF2, 15-25% of the nuclei showed no co-localization of Rad50 and TRF1. This result suggested that the association of Rad50 with telomeres is, in part, dependent on TRF2 function. These data were not extended to the TRF2^{ΔBΔM} expressing cell lines T4 and T19 (mentioned above) as the short T4/T19 telomeres yield severely reduced TRF1 signals.

TABLE 2
Effect of TRF2^{ΔBΔM} on the telomeric localization of Rad50

| | | Fraction of nuclei without | |
|---|---|---|---|
| | Fraction of cells with | telomeric Rad50^{b} | |
| | reduced telomeric TRF2^{a} | vector | TRF2^{ΔBΔM} |
| Experiment 1 | 20% | 3.6% | 15% |
| Experiment 2 | 35% | 4.0% | 24% |
| Experiment 3 | 29% | 3.6%, 5.7% | 19%, 25% |
| ^{a}Expression of TRF2^{ΔBΔM} was induced by transfection Of pTETNFlag-TRF2^{ΔBΔM} in conjunction with pUHD15-1 (van Steensel et al., 1998) in experiment 1; by using retroviral gene delivery (A. Smogorzewska and T. de Lange, unpublished) in experiment 2; or by transfection of pCDNA3-TRF2^{ΔBΔM} (J. Karlseder and T. de Lange, unpublished) in experiment 3. Cells were extracted with Triton-X-100 and | | | |
| # fixed after 24 hours and the efficiency of TRF2^{ΔBΔM} expression was determined by scoring cells that lacked telomeric TRF2 (van Steensel et al., 1998). The immunofluorescence in experiment 3 was performed in duplicate and scored in a double-blind fashion. | | | |

^{b}Cells that contained three or more overlapping punctuate signals in dual immunofluorescence with antibodies to TRF1 and Rad50 were scored positive for telomeric Rad50 staining.

Although the results suggest that TRF2 function is important for the localization of Rad50 to telomeres, there is no evidence for a direct interaction between the proteins in the Mre11 complex and TRF2. For instance, the Mre11 complex might bind to a TRF2-interacting partner such as hRap1. Human Rap1, unlike its yeast counterpart, lacks telomeric DNA binding activity and requires TRF2 to accumulate at telomeres (B. Li et al., 2000), predicting that RAP1-interacting proteins also require TRF2 function for telomeric localization.

The localization of Nbs1 in detergent extracted nuclei was distinct from the Rad50 and Mre11 patterns. In this case, the majority of interphase cells showed Nbs1 throughout the nucleus with strong signals over the nucleoli. However, a subset of the cells showed less nuclear staining and displayed Nbs1 at faint punctuate sites that represented with telomeric loci as revealed by the presence of TRF1 (FIG. 2A). The same alternate staining patterns were observed with a different anti-Nbs1 antibody (Stewart et al., 1999; Mab 1E9B4, data not shown). These data suggested that the telomeric localization of Nbs1 was confined to a minority of the cells while Rad50 and Mre11 are detectable at telomeric sites in most interphase nuclei (e.g., see Table 2). The variation in the Nbs1 localization was also observed in ALT cells lines, in which only about 10% of the cells revealed Nbs1 at TRF1 sites (FIG. 2B and data not shown).

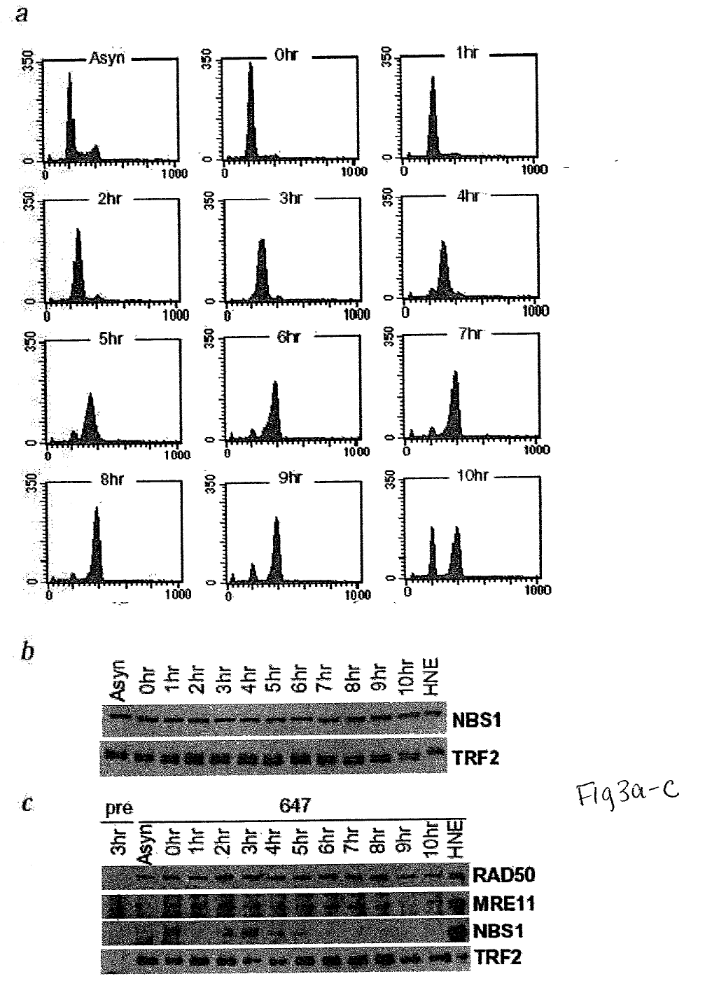

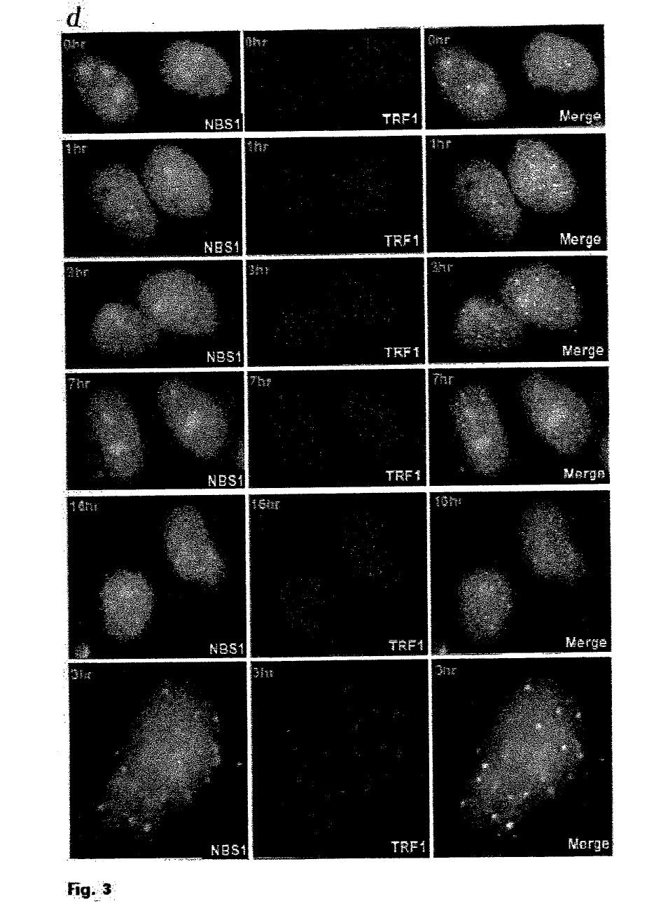

To investigate whether the association of Nbs1 with TRF2 and telomeres varied during the cell cycle, HeLa cells were arrested at G1/S using a double thymidine block followed (in some experiments) by an aphidicoline treatment. After release from the arrest, the cells progressed through S phase and G2/M synchronously as evidenced from FACS analysis (FIG. 3A). Cell extracts were examined for the association of TRF2 with Rad50, Mre11, and Nbs1. Although the steady-state levels of Nbs1 and TRF2 did not change appreciably (FIG. 3B and data not shown), there was a striking variation in the presence of Nbs1 in the TRF2 complex (FIG. 3C). Nbs1 was detected in the TRF2 complex in asynchronously growing cells and arrested cells but disappeared from the complex within 1 hour after release from the block. As cells entered and progressed through S-phase, Nbs1 reappeared in the TRF2 complex and remained associated for several hours. At the end of S-phase and in G2/M (7 hours and later), Nbs1 again disappeared from the TRF2 complex (FIG. 3C). In contrast to Nbs1, the association of Rad50 and Mre11 with TRF2 was unaltered in G1, S, and G2 (FIG. 3C).

The cell cycle dependent association of Nbs1 with TRF2 was also reflected in its telomeric localization which was obvious 3 hours after release from the thymidine/aphidicolin block when cells had entered S-phase (FIG. 3D). By contrast, cells that had progressed through most of S-phase (7 hours, FIG. 3D) had little Nbs1 at telomeres. Nbs1 was also not observed at telomeres when most of the cells had entered G1 (16 hour time point; FIG. 3D). A fraction of the cells arrested at G1/S showed Nbs1 at telomeres, consistent with the co-immunoprecipitation data (FIG. 3C). These results suggest that the interaction of Nbs1 with telomeres and TRF2 is mostly confined to S-phase.

Since the Mre11 complex has been shown to migrate to sites of DNA damage in response to γ-irradiation (Nelms et al., 1998), it was unclear whether TRF2 participates in this relocalization. Although irradiated cells showed the expected accumulation of Rad50 radiation-induced foci (FIG. 4A), the localization of TRF2 remained unaltered, with most of the protein co-localizing with TRF1 on telomeres (FIG. 4A). Furthermore, TRF2 remained associated with the Mre11 complex for up to 22 hours after γ-irradiation (FIG. 4B). These data argue against a role for TRF2 in the response to double-strand breaks induced by γ-irradiation. Furthermore, it is unlikely that mammalian telomeres function as storage sites for the repair complexes, as has been proposed for yeast (Martin et al., 1999; Mills et al., 1999; McAinsh et al., 1999), since only a minor fraction of the Mre11 complex is located at telomeres and the association with TRF2 is not disrupted by irradiation.

Rather, the presence of the Rad50/Mre11/Nbs1 complex at telomeres has implications both for the formation and function of the telomeric structure. It was recently shown that mammalian telomeres end in a large duplex loop (t-loop) (Griffith et al., 1999). Closure of the t-loop is thought to involve the invasion of the 3′ single stranded telomeric overhang into duplex telomeric sequence, creating a stable heteroduplex structure at the base of the loop. Formation of the t-loop thus resembles a DNA recombination event. The importance of the Mre11 complex for homologous recombination as well as non-homologous end joining in yeast suggests that the complex may play a structural role in DNA recombination by stabilizing chromosomal interactions (Bressan et al., 1999; Moore et al., 1996). Similarly, the human Mre11 complex may facilitate t-loop formation by stabilizing the interaction between the invading terminus and the telomeric duplex in a manner that is analogous to its role in recombinational DNA repair. Thus, t-loop formation may essentially be a specialized intrachromosomal DNA repair event mediated by the Mre11 and TRF2/hRap1 complexes. Retention of Mre11/Rad50 at the telomere may be required for the maintenance of t-loops through most of the cell cycle.

The transient recruitment of Nbs1 in S-phase, a period in which chromosome ends are replicated, suggests a role in telomere replication. Paull and Gellert (1999) recently reported that addition of Nbs1 to Rad50 and Mre11 potentiates two enzymatic activities, a DNA helicase and an endonuclease that cleaves 3′ overhangs. Addition of Nbs1 to telomeric Rad50/Mre11 might thus regulate a helicase-mediated unpairing of the t-loop base and subsequently, removal of the 3′ overhang by Mre11. This process would open the t-loop, perhaps facilitating progression of the DNA replication machinery to the end of the chromosome. Such a model also predicts that the Mre11 complex would be necessary for the release of the telomerase substrate, an accessible 3′ end, at chromosomal termini. Genetic experiments in yeast support such a role for the S. cerevisiae Mre11 complex (Nugent et al., 1998), although t-loops have not been demonstrated in yeast. Resection of the 3′ overhang by the Mre11 complex could contribute to the high rate of telomere shortening observed in primary human cells (Harley et al., 1990).

Further evidence for a role of the Mre11 complex in telomere maintenance and function is that: 1) expression of wild type Nbs1 in cells established from NBS patients (which lack Nbs1) resulted in an increased rate of telomere lengthening (data not shown), 2) chromosome analysis of a strain of mouse with a hypomorphic mutation of rad50, which mouse is severely anemic due to precocious attrition of hematopoietic stem cells and is prone to cancer, revealed frequent chromosomal fusions, indicative of telomere function, and tumors derived from these mice had abnormally long telomeres (data not shown), 3) depletion of Rad50 from murine embryonic fibroblasts resulted in rapid telomere fusion and cell death (data not shown), and 4) a cross between mice carrying a hypomorphic mutation of NBS1 and DNAPK mutant mice resulted in severe ranting, and complete failure to develop germ cells (data not shown). These phenotypic outcomes are all hallmarks of disrupted telomere function (data not shown). Moreover, cell lines established from the NBS/DNAPK double mutants undergo premature senescence. These observations underscore the importance of the Mre11 complex for telomere function, the utility of the NBS mutant mouse for telomere analysis, the development and testing of compounds perturbing Mre11 complex functions, and the utility of reagents to inhibit Nbs1 function and interaction with Mre11 at telomeres.

### REFERENCES

Ajimura et al., Genetics 133:51-66 (1993).

Baumann et al., Cell, 87:757 (1996).

Bigbee et al., Am. J. Hum. Genet., 44:402-408 (1989).

Bilaud et al., Nature Genetics, 17, 236-239 (1997).

Bischoff et al., EMBO J. 17, 3052-3065 (1998).

Blocher et al., Int. J. Radiat. Biol., 60:791-802 (1991).

Bressan et al., Mol. Cell. Biol., 19, 7681-7687 (1999).

Broccoli et al., Nature Gen. 17, 231-235 (1997).

Bryan et al., Nat. Med. 3, 1271-1274 (1997).

Bubley et al., Somat. Cell Mol. Genet., 13, 111-7 (1987).

Carney et al., Cell 93, 477-486 (1998).

Chaganti et al., Proc. Natl. Acad. Sci. USA, 71:4508-4512 (1974).

Chong et al., Science, 270, 1663-1667 (1995).

Cleaver, Birth Defects, 25:61-82 (1989).

Connelly et al., J. Biol. Chem., 272:19819-26 (1997).

Connelly et al., Genes to Cells, 1:285-291 (1996).

Comforth et al., Science, 227:1589-1591. (1985).

Dignam et al., Nucl. Acids Res., 11, 1475-1489 (1983).

Dionne, Nucleic Acids Res., 26, 5365-5371 (1998).

Dolganov et al., Mol. Cell. Biol. 16:4832-4841 (1996).

Durfee et al., Genes & Dev., 7:555-569 (1993).

Ellis, N. A., Curr. Opin. Genet. Dev. 7:354-63 (1997).

Eng et al., J. Am. Soc. Mass Spectrom., 5:976-989 (1994).

Fukuchi et al., Proc. Natl. Acad. Sci. USA 86:5893-5897 (1989).

Game et al., Cancer Biol., 4:73-83 (1993).

Gatlin et al., Anal. Biochem. (1998). Submitted.

Gatti et al., Medicine, 70:99-117 (1991).

Gellert, Adv. Immunol., 64:39-64 (1997).

German, In Chromosome Mutation & Neoplasia, J. German, ed. (New York: Alan R. Liss), pp. 11-21.

Gibson et al., J. Bacteriol., 174:1222-1228 (1992).

Griffith et al., Cell, 97, 503-514 (1999).

Gupta et al., Proc. Natl. Acad. Sci. U.S.A., 94:463-68 (1997).

Haber, Cell, 95, 583-586 (1998).

Harlow et al., A laboratory manual (Cold Spring Harbor: Cold Spring Harbor Publications) (1988).

Harley et al., Nature, 345, 458-460 (1990).

Hoekstra, Curr. Opin. Genet. Dev., 7:170-5 (1997).

Hojo et al., Mut. Res., 334:59-69 (1995).

Hsieh et al., J. Biol. Chem., 268:20105-20109 (1993).

Ivanov et al., Genetics, 142:693-704 (1996).

Ivanov et al., Mol. Cell. Biol., 14:3414-25 (1994).

Jackson, Cancer Surv., 28:261-279 (1996).

Jackson et al., Trends Biochem. Sci., 20:412-415 (1995).

James et al., Genetics, 144:1425-36 (1996).

Jayakumar et al., Proc. Natl Acad. Sci. USA, 93:14509-14 (1996).

Johzuka et al., Genetics, 139:1521-1532 (1995).

Jongmans et al., Mol. Cell. Biol. 17:5016-22 (1997).

Jung et al., Cancer Res., 57:24-27 (1997).

Karlseder et al., Science, 283, 1321-1325 (1999).

Keegan et al., Genes Dev., 10:2423-2437 (1996).

Kolodner, Trends Biochem. Sci. 20:397-401 (1995).

Langlois et al., Proc. Natl. Acad. Sci. USA, 86:670-674 (1989).

Li et al., Cell Prolif., 28:571-79 (1995).

Li et al., Cell, 101:471-483 (2000).

Link et al., Electrophoresis, 18:1314-34 (1997).

Luo et al., Proc. Natl. Acad. Sci., 96, 7376-7381 (1999).

MacKay et al., Med. Phys., 1998.

Mann et al., Anal. Chem., 68, 4390-4399 (1994).

Martin et al., Cell, 97, 621-633 (1999).

Maser et al., Mol. Cell. Biol., 17, 6087-6097 (1997).

Matsuura et al., Am. J. Hum. Genet. 60:1487-94 (1997).

McAinsh et al., Curr. Biol., 9, 963-966 (1999).

Meyn, Cancer Res., 55:5991-6001 (1995).

Meyn, Science, 260:1327-1330 (1993).

Mills et al., Cell 97, 621-633 (1999).

Moore et al., Mol. Cell. Biol., 16, 2164-2173 (1996).

Moreau et al., Cell Biol. 19, 556-566 (1999).

Murnane, Cancer Met. Rev., 14:17-29 (1995).

Murti et al., Proc. Natl. Acad. Sci. USA, 96, 14436-14439 (1999).

Nelms et al., Radiat. Res., 1998.

Nelms et al., Science, 280, 590-592 (1998).

Nevaldine et al., Radiat. Res., 133:370-374 (1993).

Nishida et al., J. Biol. Chem., 263:501-10 (1988).

Nugent et al., Curr. Biol. 8, 657-660 (1998).

Ogawa et al., Adv. Biophys., 31:67-76 (1995).

Pandita et al., Radiat. Res., 130:94-103 (1992).

Paull et al., Genes & Dev. 13, 1276-1288 (1999).

Perez-Vera et al., Am. J. Med. Genet., 70:24-7 (1997).

Petrini et al., Am. J. Hum. Genet., 64, 1264-1269 (1999).

Petrini et al., J. Immunol., 152:176-183 (1994).

Petrini et al., Genomics, 29:80-86 (1995).

Petrini et al, Semin. Immunol., 9:181-8 (1997).

Rabbitts et al., Nature, 372:143-9 (1994).

Rowley, Leukemia, S1-6 (1994).

Rowley et al., Proc. Natl. Acad. Sci. USA, 87:9358-9362 (1990).

Saar et al., Am. J. Hum. Genet., 60:605-10 (1997).

Savitsky et al., Science 268:1749-1753 (1995).

Scully et al., Cell, 88:265-75 (1997).

Scully et al., Cell, 90:425-35 (1997).

Sharples et al., Mol. Microbiol., 17:1215-1217 (1995).

Shevchenko et al., Anal. Chem., 68:850-858 (1996).

Shevchenko et al., Rapid Commun. Mass. Spectrom., 11, 1015-1024 (1997).

Shiloh, Annu. Rev. Genet., 31:635-62 (1997).

Smith et al., Science, 282, 1484-1487 (1998).

Smogorzewska et al., Mol. Cell. Biol., 20, 1659-1668 (2000).

Stavnezer, Adv. Immunol., 61:79-146 (1996).

Stewart et al., Cell, 99, 577-587 (1999).

Stumm et al., Am. J. Hum. Genet., 60:1246-51 (1997).

Sullivan et al., Clin. Immunol. Immunopathol., 82:43-8 (1997).

Sung et al., Cell, 82:453-61 (1995).

Taalman et al., Mut. Res., 112:23-32 (1983).

Timme et al., Amer. J. Med. Sci., 295:40-48 (1988).

Tsubouchi et al., Mol. Cell. Biol., 18:260-8 (1998).

Usui et al., Cell 95 705-716 (1998).

van der Burgt et al., J. Med. Genet. 33:153-6 (1996).

van Steensel et al., Cell, 92, 401-413 (1998).

van Steensel et al., Nature, 385, 740-743 (1997).

Weemaes et al., Acta. Paediatr. Scand., 70:557-64 (1981).

Weemaes et al., Int. J. Radiat. Biol, 66:S185-8 (1994).

Wilm et al., Nature, 379, 466-469 (1996).

Xiao et al., Nucleic Acids Res., 25:2985-91 (1997).

Yates et al., Anal. Chem., 67:3203-3210 (1995).

Yeager et al., Cancer Res., 59, 4175-4179 (1999).

Young et al., Hum. Genet., 82:113-7 (1989).

Zhong et al., Mol. Cell. Biol., 13, 4834-4843 (1992).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to identify an agent that modulates telomere length or maintenance associated with the mammalian Mre11 complex, comprising:
a) contacting an agent with a mammalian cell comprising two or more proteins of the Mre11 complex, a subcellular fraction of a mammalian cell comprising two or more proteins of the Mre11 complex, an extract of a mammalian cell comprising two or more proteins of the Mre11 complex, or a composition comprising two or more isolated proteins of the mammalian Mre11 complex, any of which comprise DNA comprising a telomere; and
b) detecting or determining whether the agent modulates mammalian Mre11 complex formation, amount or activity at telomeres.

2. An agent identified by the method of claim 1.

3. A method to modulate telomere length or maintenance, comprising:
contacting a mammalian cell comprising two or more proteins of the Mre11 complex, a subcellular fraction of a mammalian cell comprising two or more proteins of the Mre11 complex, an extract of a mammalian cell comprising two or more proteins of the Mre11 complex, or a composition comprising two or more isolated proteins of the Mre11 complex, any of which comprise DNA comprising a telomere, with an agent in an amount effective to modulate Mre11 complex formation, amount or activity at telomeres.

4. The method of claim 1 or 3 wherein the agent modulates the amount or activity of p95.

5. The method of claim 1 or 3 wherein the agent modulates the amount or activity of Mre11.

6. The method of claim 1 or 3 wherein the agent modulates the amount or activity of Rad50.

7. The method of claim 1 or 3 wherein the agent is an antibody.

8. The method of claim 1 or 3 wherein the agent inhibits Mre11 complex amount, formation or activity.

9. The method of claim 1 or 3 wherein the agent enhances Mre11 complex amount, formation or activity.

10. The method of claim 1 or 3 wherein the modulation of the Mre11 complex inhibits the proliferation of the cell.

11. The method of claim 1 or 3 wherein the agent enhances the amount or activity of p95.

12. The method of claim 1 or 3 wherein the agent decreases telomere length.

13. The method of claim 1 or 3 wherein the agent increases telomere length.

14. The method of claim 1 or 3 wherein the cell is from a mammal that has reduced levels of p95 relative to the level of p95 in a wild-type mammal.

15. The method of claim 1 or 3 wherein the cells is from a mammal that has reduced levels of Rad50 relative to the level of Rad50 in a wild-type mammal.

16. A method to detect a mammalian cell in S phase, comprising:
(a) contacting a mammalian cell comprising two or more proteins of the Mre11 complex, a subcellular fraction of a mammalian cell comprising two or more proteins of the Mre11 complex, an extract of a mammalian cell comprising two or more proteins of the Mre11 complex, any of which comprise DNA comprising a telomere, with an agent that binds p95 so as to form a complex; and
(b) detecting or determining whether the complex is associated with TRF2 at a telomere.

17. The method of claim 16 wherein the agent is an antibody.

18. The method of claim 16 wherein the telomere is detected with an agent that binds TRF2.
